(19) United States
(12) Patent Application Publication
(10) Pub. No.: US 2024/0023844 A1
(43) Pub. Date: Jan. 25, 2024

(54) INFANT BLOOD OXYGEN SATURATION MONITORING METHOD AND INTELLIGENT MONITORING DEVICE

(71) Applicant: SHENZHEN KYLINCARELIFE MEDICAL TECHNOLOGY CO., LTD, Guangdong (CN)

(72) Inventor: Shiqing JI, Shenzhen, Guangdong (CN)

(21) Appl. No.: 18/273,317

(22) PCT Filed: Jan. 11, 2022
(86) PCT No.: PCT/CN2022/071402

§ 371 Date: Jul. 20, 2023

(30) Foreign Application Priority Data
Jan. 20, 2021 (CN) 202110078606.7

(51) Int. Cl. A61B 5/1455; A61B 5/00

(52) U.S. Cl. CPC A61B 5/14552; A61B 5/7278; A61B 2503/04; A61B 5/0261

(57) ABSTRACT

The present disclosure provides an infant blood oxygen saturation monitoring method and an intelligent monitoring device. The infant blood oxygen saturation monitoring method includes the following steps: A, performing reflective testing on soles of an infant by red light λ₁ and infrared light λ₂ to obtain blood flow signals I_{MAX}^{λ1} and I_{MAX}^{λ2} at the soles of the infant, respectively; B, analyzing the blood flow signals to obtain alternating current components I_{AC}^{λ1} and I_{AC}^{λ2} of the blood flow signals, respectively; C, analyzing intensity changes of the red light and the infrared light, and a relationship between blood flow signals and a blood oxygen saturation, and calculating test constants As, Bs, and Cs; and D, combining As, Bs, and Cs with I_{MAX}^{λ1}, I_{MAX}^{λ2}, I_{AC}^{λ1}, and I_{AC}^{λ2}, and obtaining the blood oxygen saturation of the infant by a blood oxygen saturation calculation formula X:

$${SpO}_{2} = As - {B}_{s} \cdot \frac{{I}_{AC}^{{λ}_{1}} / {I}_{MAX}^{{λ}_{1}}}{{I}_{AC}^{{λ}_{2}} / {I}_{MAX}^{{λ}_{2}}} + {{C}_{s} ( \frac{{I}_{AC}^{{λ}_{1}} / {I}_{MAX}^{{λ}_{1}}}{{I}_{AC}^{{λ}_{2}} / {I}_{MAX}^{{λ}_{2}}} )}^{2} .$$

### CROSS REFERENCE TO RELATED APPLICATION

This patent application is a national stage application of International Patent Application No. PCT/CN2022/071402, filed on Jan. 11, 2022, which claims the benefit and priority of Chinese Patent Application No. 202110078606.7, filed with the China National Intellectual Property Administration on Jan. 20, 2021, the disclosure of which is incorporated by reference herein in its entirety as part of the present application.

### TECHNICAL FIELD

The present disclosure relates to the technical field of blood oxygen detection, and in particular to an infant blood oxygen saturation monitoring method and an intelligent monitoring device.

### BACKGROUND

At present, most products on the market are transmissive blood oxygen and pulse monitors which are generally applicable to children and adults. A measuring position is often a finger. There are also some reflective blood oxygen and pulse monitoring devices on the market, which are all applicable to adults or children. At present, several universal blood oxygen monitoring instruments provided by companies such as HealForce, CONTEC, and EDAN use strap-on probes which needs to be strapped tightly so as to provide stable readings. Even though an infant's crying and struggling due to strapping are neglected, the reading of a strap-on probe is highly unstable when the infant moves, and a false alarm may be given. A wired connection between the probe and a host is not safe. Meanwhile, the strapped position of the infant may become red and swollen due to long-time compression. The strapped position needs to be changed to a leg every two to three hours to prevent pressure sores. Thus, higher requirements are put forward on the wearability and comfort of the products.

The present disclosure provides an intelligent sole blood oxygen and pulse monitoring device and method applicable to infants at the age of 0 to 5 years. There are no relevant products for the age group and the measuring position on the market. Due to the particularities of infants at the age of 0 to 5 years in terms of measuring position, blood signal strength, use and wearing manner, and the like, such as the characteristics of thin sole skin, good permeability, and rich blood vessels, higher requirements are put forward on the intelligent sole blood oxygen and pulse monitoring device applicable to infants at the age of 0 to 5 years in terms of use manner, wearing manner, wearability, signal acquisition, blood oxygen and pulse accuracy, and the like.

Therefore, there is an urgent need on the market for a method capable of accurately and stably monitoring an infant blood oxygen saturation.

### SUMMARY

In view of the problems of the prior art, the present disclosure provides an infant blood oxygen saturation monitoring method and an intelligent monitoring device that can accurately measure an infant's blood oxygen saturation on the premise of reducing discomfort of the infant as much as possible.

To solve the above-mentioned technical problems, the present disclosure adopts the following technical solutions:

An infant blood oxygen saturation monitoring method includes the following steps:
A, performing reflective testing on soles of an infant by red light λ₁ and infrared light λ₂ to obtain blood flow signals I_{MAX}^{λ1} and I_{MAX}^{λ2} at the soles of the infant, respectively;
B, analyzing the blood flow signals to obtain alternating current components I_{AC}^{λ1} and I_{AC}^{λ2} of the blood flow signals, respectively;
C, analyzing intensity changes of the red light and the infrared light, and a relationship between blood flow signal strength and a blood oxygen saturation, and calculating test constants As, Bs, and Cs;
D, combining As, Bs, and Cs with I_{MAX}^{λ1}, I_{MAX}^{λ2}, I_{AC}^{λ1}, and I_{AC}^{λ2}, and obtaining the blood oxygen saturation of the infant by a blood oxygen saturation calculation formula X:

$${SpO}_{2} = As - {B}_{s} \cdot \frac{{I}_{AC}^{{λ}_{1}} / {I}_{MAX}^{{λ}_{1}}}{{I}_{AC}^{{λ}_{2}} / {I}_{MAX}^{{λ}_{2}}} + {{C}_{s} ( \frac{{I}_{AC}^{{λ}_{1}} / {I}_{MAX}^{{λ}_{1}}}{{I}_{AC}^{{λ}_{2}} / {I}_{MAX}^{{λ}_{2}}} )}^{2} .$$

Further, the test constants As, Bs, and Cs may be calculated by the following steps:
C1, obtaining test data of a direct current component and an alternating current component corresponding to a blood oxygen value of 60 to 100 by a blood oxygen saturation calibrator; and
C2, in combination with clinical experience of a correspondence between blood oxygen values and blood flow signals at soles of an infant, calculating the As, Bs, and Cs by combined polynomial fitting and nonlinear least square fitting.

Further, in step B, I_{AC}^{λ1} and I_{AC}^{λ2} may be calculated by:

I_{AC}^{λ1}=I_{PAC}^{λ1}=(I_{MAXAC}^{λ1}+I_{MINAC}^{λ1})/2; and

I_{AC}^{λ2}=I_{PAC}^{λ2}=(I_{MAXAC}^{λ2}+I_{MINAC}^{λ2})/2;
where I_{MAXAC}^{λ1} represents a maximum of an alternating current signal I_{DBAC}^{λ1} of a light signal with a wavelength of λ1 after being subjected to adaptive weighting and windowing and envelope detection;
I_{MINAC}^{λ1} represents a minimum of the alternating current signal I_{DBAC}^{λ1} of the light signal with the wavelength of λ1 after being subjected to adaptive weighting and windowing and envelope detection;
I_{MAXAC}^{λ2} represents a maximum of an alternating current signal I_{DBAC}^{λ2} of a light signal with a wavelength of λ2 after being subjected to adaptive weighting and windowing and envelope detection; and
I_{MINAC}^{λ2} represents a minimum of the alternating current signal I_{DBAC}^{λ2} of the light signal with the wavelength of λ2 after being subjected to adaptive weighting and windowing and envelope detection.

Still further, in step B, I_{DBAC}^{λ1} and I_{DBAC}^{λ2} may be specifically calculated by the following steps:
B1, according to characteristics of the soles of an infant, sampling, as a basis, data of 30 pulse periods by the red light and the infrared light, respectively, with a sampling frequency of 100 Hz, to obtain about 3000 sample points I_{DBAC}^{λ1}[i], where i represents a serial number of a sample point, and i=1 to 3000; and
B2, substituting the data of the sample points into the following formula Y: RC*(I_{DBAC}^{λ1}[i]−I_{DBAC}^{λ1}[i−1])/Δt, to obtain I_{DBAC}^{λ1}[i]=I_{DBAC}^{λ1} [i−1]*[K/(K+1)], where K=RC/Δt.

Further, in the formula X, values of the blood flow signals I_{MAX}^{λ1} and I_{MAX}^{λ2} may be replaced by direct current components I_{DC}^{λ1} and I_{DC}^{λ2} of the blood flow signals of λ1 and λ2, respectively, and calculated respectively by:

I_{DC}^{λ1}=I_{MAX}^{λ1}−I_{AC}^{λ1}; and

I_{DC}^{λ2}=I_{MAX}^{λ2}−I_{AC}^{λ2}.

Further, after calculating the blood oxygen saturation of the infant by the formula, at least the following steps may be performed:
E, determining a sample size of blood oxygen saturations of infants, and sample distribution and analysis data by statistical software according to clinical characteristics of the soles of infants and in combination with multi-center test requirements of clinical tests and linear regression in statistics; and
F, performing a normality test on the sample distribution and calculating a skewness.

The present disclosure provides an intelligent monitoring device using the infant blood oxygen saturation monitoring method described above, including a housing, and a thermometer and a blood oxygen heart rate monitor that are capable of being accommodated in the housing, where the thermometer is configured to monitor a body temperature under an armpit of an infant; the blood oxygen heart rate monitor is configured to be placed on a sole of the infant to monitor a blood oxygen heart rate; the housing is provided with a main control unit configured for signal interaction with the outside; and the thermometer and the blood oxygen heart rate monitor are in signal connection with the main control unit.

The present disclosure has the following beneficial effects: the present disclosure provides an infant blood oxygen saturation monitoring method. A blood oxygen heart rate monitor is configured to acquire blood flow signals at the soles of an infant by using the monitoring method, and a blood oxygen saturation of the infant is calculated by a formula. Compared with the prior art, discomfort of the infant during monitoring can be reduced. A thermometer is configured to intelligently measure a body temperature under an armpit. Simultaneous monitoring of the body temperature and the blood oxygen heart rate is realized. Moreover, the accuracy and stability of monitoring are improved.

### BRIEF DESCRIPTION OF THE DRAWINGS

FIG. 1 is a schematic diagram of Example 2; and

FIG. 2 is a circuit block diagram of Example 2.

List of Reference Numerals: 1—housing, 2—thermometer, and 3—blood oxygen heart rate monitor.

### DETAILED DESCRIPTION OF THE EMBODIMENTS

To facilitate the understanding by those skilled in the art, the present disclosure will be further described with reference to the examples and the accompanying drawings. The contents mentioned in the embodiments are not intended to limit the present disclosure. The present disclosure is described in detail below with reference to the accompanying drawings.

### Example 1

The present example provides an infant blood oxygen saturation monitoring method, including the following steps:
A, perform reflective testing on soles of an infant by red light λ₁ and infrared light λ₂ to obtain blood flow signals I_{MAX}^{λ1} and I_{MAX}^{λ2} at the soles of the infant, respectively;
B, analyze the blood flow signals to obtain alternating current components I_{AC}^{λ1} and I_{AC}^{λ2};
C, analyze intensity changes of the red light and the infrared light, and a relationship between blood flow signal strength and a blood oxygen saturation, and calculating test constants As, Bs, and Cs;
D, combine As, Bs, and Cs with I_{MAX}^{λ1}, I_{MAX}^{λ2}, I_{AC}^{λ1}, and I_{AC}^{λ2}, and obtaining the blood oxygen saturation of the infant by a blood oxygen saturation calculation formula X:

$${SpO}_{2} = As - {B}_{s} \cdot \frac{{I}_{AC}^{{λ}_{1}} / {I}_{MAX}^{{λ}_{1}}}{{I}_{AC}^{{λ}_{2}} / {I}_{MAX}^{{λ}_{2}}} + {{C}_{s} ( \frac{{I}_{AC}^{{λ}_{1}} / {I}_{MAX}^{{λ}_{1}}}{{I}_{AC}^{{λ}_{2}} / {I}_{MAX}^{{λ}_{2}}} )}^{2} .$$

Absorption characteristics of oxyhemoglobin (Hb02) and hemoglobin (HbR) to the red light and the infrared light have a significant difference: Hb02 absorbs the red light with a wavelength of 600 nm to 700 nm; and HbR absorbs the infrared light with a wavelength of 800 nm to 1000 nm. According to this principle, the infrared light and the red light are emitted onto the soles of the infant to obtain the blood flow signals of the two lights by reflective testing. The alternating current components I_{AC}^{λ1} and I_{AC}^{λ2} and direct current components are then obtained by signal analysis. Since the alternating current component is generally only 1% of the direct current component, to simplify calculation, the direct current components may be considered as equal to the blood flow signals I_{MAX}^{λ1} and I_{MAX}^{λ2}. The measured data is then substituted into the above formula to obtain real-time blood oxygen saturation data of the infant.

Compared with the prior art, by performing blood oxygen saturation monitoring on the soles of the infant, acquiring the blood flow signals at the soles of the infant by using a blood oxygen heart rate monitoring, and calculating the blood oxygen saturation of the infant by the formula, the discomfort of the infant can be reduced, and an interference with the monitoring data caused by fidgeting or crying of the infant when the infant feels uncomfortable can be avoided.

Moreover, in another aspect, calculation by the above formula is performed particularly for the blood flow characteristic of the soles of the infant. The above formula may be used as an empirical formula for a linear relationship of blood oxygen saturation measurement. That is, an accurate blood oxygen saturation of the infant can be obtained by performing calculation parameters collected at the soles of the infant by the above formula.

In this example, the test constants As, Bs, and Cs are calculated by the following steps:
C1, obtain test data of a direct current component and an alternating current component corresponding to a blood oxygen value of 60 to 100 by a blood oxygen saturation calibrator; and
C2, in combination with clinical experience of a correspondence between blood oxygen values and blood flow signals at soles of an infant, calculate the As, Bs, and Cs by combined polynomial fitting and nonlinear least square fitting.

In the application of the formula, As, Bs, and Cs are obtained by sampling blood oxygen values of the soles of infants with a sample size of 100 and performing calculation according to sampling results. In the formula X, As is used to adjust the direct current component generated by the hemoglobin in the arterial blood flow of the sole of the infant; a combination of Bs and a sampling current is used to adjust a venous blood interference, interferences generated by human body tissues in transmission paths of the red light and the infrared light, a mutual movement interference, an external light interference, a circuit interference, and the like; and a combination of Cs and a sampling current is used as a signal indicating generation of the oxygen and the hemoglobin of a human body. Interferences encountered when monitoring can be eliminated during calculation by means of the above formula and the measured data to guarantee the accuracy of the obtained result.

In practical use, to eliminate some monitoring results in special cases (e.g., an intelligent monitoring device is displaced from the sole of the infant), in the present example, after calculating the blood oxygen saturation of the infant by the formula, at least the following steps are performed:
G, determine a sample size of blood oxygen saturations of infants, and sample distribution and analysis data by statistical software according to clinical characteristics of the soles of infants and in combination with multi-center test requirements of clinical tests and linear regression in statistics, where the statistical software is preferably statistic package for social science (SPSS); and
H, perform a normality test on the sample data distribution and calculate a skewness.

In the present example, the wavelength of the red light λ₁ is between 600 nm and 650 nm, and the wavelength of the infrared light λ₂ is between 900 nm and 950 nm.

In the present example, in step A, an intelligent monitoring device is in contact with the soles of the infant to carry out the reflective testing. That is, the intelligent monitoring device is directly in contact with the soles of the infant and then emits the red light and the infrared light for data acquisition. The influence of other interferences than those in the body of the infant on the monitoring data can be effectively reduced. Subsequently, corresponding noise can be eliminated by means of a related filter circuit and the formula X.

By introducing new parameters, more accurate and reliable monitoring effect can be achieved. The infant blood oxygen saturation monitoring method is applicable to perform blood oxygen saturation monitoring on infants of different body constitutions.

As a matter of course, in the above formula X, to improve the accuracy of blood oxygen detection, values of the blood flow signals I_{MAX}^{λ1} and I_{MAX}^{λ2} may be replaced by direct current components I_{DC}^{λ1} and I_{DC}^{λ2} of the blood flow signals of λ₁ and λ₂, respectively, and calculated respectively by:

I_{DC}^{λ1}=I_{MAX}^{λ1}−I_{AC}^{λ1}; and

I_{DC}^{λ2}=I_{MAX}^{λλ2}−I_{AC}^{λ2}.

The improvement of the accuracy can be applied to a device and a test having strict monitoring data requirements, thereby improving the accuracy of results.

As a preferred solution of the present example, in step B,

I_{AC}^{λ1}=I_{PAC}^{λ1}=(I_{MAXAC}^{λ1}+I_{MINAC}^{λ1})/2; and

I_{AC}^{λ2}=I_{PAC}^{λ2}=(I_{MAXAC}^{λ2}+I_{MINAC}^{λ2})/2;
where I_{MAXAC}^{λ1} represents a maximum of an alternating current signal I_{DBAC}^{λ1} of a light signal with a wavelength of λ1 after being subjected to adaptive weighting and windowing and envelope detection;
I_{MINAC}^{λ1} represents a minimum of the alternating current signal I_{DBAC}^{λ1} of the light signal with the wavelength of λ1 after being subjected to adaptive weighting and windowing and envelope detection;
I_{MAXAC}^{λ2} represents a maximum of an alternating current signal I_{DBAC}^{λ2} of a light signal with a wavelength of λ2 after being subjected to adaptive weighting and windowing and envelope detection; and
I_{MINAC}^{λ2} represents a minimum of the alternating current signal I_{DBAC}^{λ2} of the light signal with the wavelength of λ2 after being subjected to adaptive weighting and windowing and envelope detection.

Preferably, in step B, I_{DBAC}^{λ1} and I_{DBAC}^{λ2} are specifically calculated by the following steps:
B1, according to characteristics of the soles of an infant, sample, as a basis, data of 30 pulse periods by the red light and the infrared light, respectively, with a sampling frequency of 100 Hz, to obtain about 3000 sample points I_{DBAC}^{λ1}[i], where i represents a serial number of a sample point, and i=1 to 3000; and
B2, substitute the data of the sample points into the following formula Y: RC*(I_{DBAC}^{λ1}[i]−I_{DBAC}^{λ1}[i−1])/Δt, to obtain I_{DBAC}^{λ1}[i]=I_{DBAC}^{λ1}[i−1]*[K/(K+1)], where K=RC/Δt.

In the above calculation, 30 pulse periods are used as a basis, and data of the red light and the infrared data are acquired at the soles of the infant. Thus, maximums and minimums are picked out of 3000 sample points for calculation, guaranteeing the reliability of the data used in the formula calculation in the present disclosure.

### Example 2

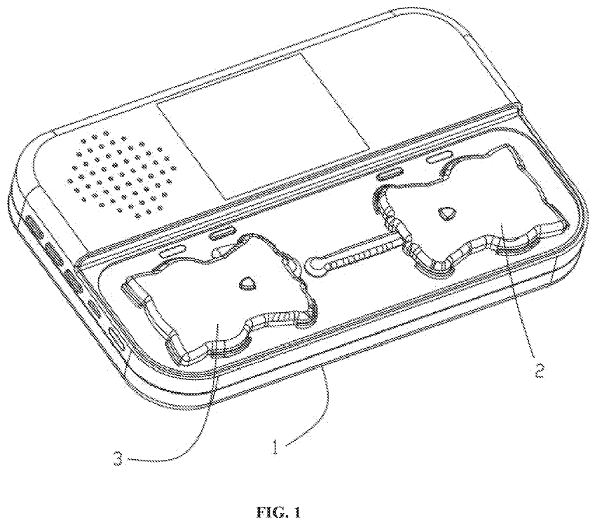

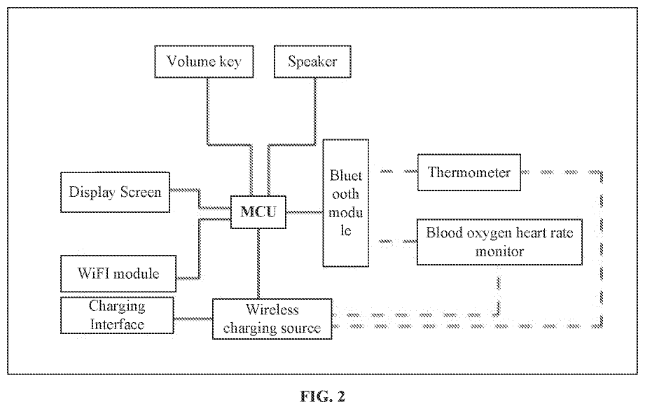

As shown in FIG. 1 and FIG. 2, the present disclosure provides an intelligent monitoring device. The intelligent monitoring device using the method of Example 1 and includes a housing 1, and a thermometer 2 and a blood oxygen heart rate monitor 3 that are capable of being accommodated in the housing 1. The thermometer 2 is configured to monitor a body temperature under an armpit of an infant. The blood oxygen heart rate monitor 3 is configured to be placed on a sole of the infant to monitor a blood oxygen heart rate. The housing 1 is provided with a main control unit configured for signal interaction with a cloud server. The thermometer 2 and the blood oxygen heart rate monitor 3 are in signal connection with the main control unit by Bluetooth

In use, the thermometer 2 or the blood oxygen heart rate monitor 3 are taken out of an accommodating box of the housing 1. When measuring the blood oxygen heart rate, the blood oxygen heart rate monitor 3 is placed on the sole of the infant. The blood oxygen heart rate monitor 3 may be relatively fixed to the sole of the infant by means of a sock. The Hb02 and HbR signals of the infant are acquired by the blood oxygen heart rate monitor 3 and then amplified, and processed by the method of Example 1 to obtain the blood oxygen saturation of the infant. When measuring the body temperature, the thermometer 2 is directly in contact with an armpit of the infant to measure the temperature. Thus, simultaneous intelligent monitoring of the body temperature and the blood oxygen pulse of the infant is realized. After the data of the blood oxygen saturation and the body temperature is monitored, the data is transmitted to the main control unit for being displayed or uploaded by the main control unit, facilitating viewing by a user. Compared with the prior art in which a device is directly strapped on an infant, the present disclosure realizes intelligent monitoring by placing the blood oxygen heart rate monitor on the sole of the infant and putting the thermometer under the armpit. The discomfort of the infant is reduced and the influence of fidgeting of the infant on the monitoring effect is avoided.

The foregoing are merely descriptions of the preferred embodiments of the present disclosure and are not intended to limit the present disclosure in any form. Although the present disclosure has been disclosed above by the preferred embodiments, these embodiments are not intended to limit the present disclosure. Any person skilled in the art may make some changes or modifications to implement equivalent embodiments with equivalent changes by using the technical contents disclosed above without departing from the scope of the technical solutions of the present disclosure. Any simple modification, equivalent change and modification made to the foregoing embodiments according to the technical essence of the present disclosure without departing from the contents of the technical solutions of the present disclosure shall fall within the scope of the technical solutions of the present disclosure.

1. An infant blood oxygen saturation monitoring method, comprising the following steps:
A, performing reflective testing on soles of an infant by red light λ₁ and infrared light λ₂ to obtain blood flow signals I_{MAX}^{λ1} and I_{MAX}^{λ2} at the soles of the infant, respectively;
B, analyzing the blood flow signals to obtain alternating current components I_{AC}^{λ1} and I_{AC}^{λ2} of the blood flow signals, respectively;
C, analyzing intensity changes of the red light and the infrared light, and a relationship between blood flow signal strength and a blood oxygen saturation, and calculating test constants As, Bs, and Cs;
D, combining As, Bs, and Cs with I_{MAX}^{λ1}, I_{MAX}^{λ2}, I_{AC}^{λ1}, and I_{AC}^{λ2}, and obtaining the blood oxygen saturation of the infant by a blood oxygen saturation calculation formula X:
$${SpO}_{2} = As - {B}_{s} \cdot \frac{{I}_{AC}^{{λ}_{1}} / {I}_{MAX}^{{λ}_{1}}}{{I}_{AC}^{{λ}_{2}} / {I}_{MAX}^{{λ}_{2}}} + {{C}_{s} ( \frac{{I}_{AC}^{{λ}_{1}} / {I}_{MAX}^{{λ}_{1}}}{{I}_{AC}^{{λ}_{2}} / {I}_{MAX}^{{λ}_{2}}} )}^{2} .$$

2. The infant blood oxygen saturation monitoring method according to claim 1, wherein the test constants As, Bs, and Cs are calculated by the following steps:
C1, obtaining test data of a direct current component and an alternating current component corresponding to a blood oxygen value of 60 to 100 by a blood oxygen saturation calibrator; and
C2, in combination with clinical experience of a correspondence between blood oxygen values and blood flow signals at soles of an infant, calculating As, Bs, and Cs by combined polynomial fitting and nonlinear least square fitting.

3. The infant blood oxygen saturation monitoring method according to claim 1, wherein in step B, I_{AC}^{λ1} and I_{AC}^{λ2} are calculated by:
I_{AC}^{λ1}=I_{PAC}^{λ1}=(I_{MAXAC}^{λ1}+I_{MINAC}^{λ1})/2; and
I_{AC}^{λ2}=I_{PAC}^{λ2}=(I_{MAXAC}^{λ2}+I_{MINAC}^{λ2})/2;
wherein I_{MAXAC}^{λ1} represents a maximum of an alternating current signal I_{DBAC}^{λ1} of a light signal with a wavelength of λ1 after being subjected to adaptive weighting and windowing and envelope detection;
I_{MINAC}^{λ1} represents a minimum of the alternating current signal I_{DBAC}^{λ1} of the light signal with the wavelength of λ1 after being subjected to adaptive weighting and windowing and envelope detection;
I_{MAXAC}^{λ2} represents a maximum of an alternating current signal I_{DBAC}^{λ2} of a light signal with a wavelength of λ2 after being subjected to adaptive weighting and windowing and envelope detection; and
I_{MINAC}^{λ2} represents a minimum of the alternating current signal I_{DBAC}^{λ2} of the light signal with the wavelength of λ2 after being subjected to adaptive weighting and windowing and envelope detection.

4. The infant blood oxygen saturation monitoring method according to claim 3, wherein in step B, I_{DBAC}^{λ1} and I_{DBAC}^{λ2} are specifically calculated by the following steps:
B1, according to characteristics of the soles of an infant, sampling, as a basis, data of 30 pulse periods by the red light and the infrared light, respectively, with a sampling frequency of 100 Hz, to obtain about 3000 sample points I_{DBAC}^{λ1}[i], wherein i represents a serial number of a sample point, and i=1 to 3000; and
B2, substituting the data of the sample points into the following formula Y: RC*(I_{DBAC}^{λ1}[i]−I_{DBAC}^{λ1}[i−1])/Δt, to obtain I_{DBAC}^{λ1}[i]=I_{DBAC}^{λ1}[i−1]*[K/(K+1)], wherein K=RC/Δt.

5. The infant blood oxygen saturation monitoring method according to claim 1, wherein in the formula X, values of the blood flow signals I_{MAX}^{λ1} and I_{MAX}^{λ2} are replaceable by direct current components I_{DC}^{λ1} and I_{DC}^{λ2} of the blood flow signals of λ1 and λ2, respectively, and calculated respectively by:
I_{DC}^{λ1}=I_{MAX}^{λ1}−I_{AC}^{λ1}; and
I_{DC}^{λ2}=I_{MAX}^{λ2}−I_{AC}^{λ2}.

6. The infant blood oxygen saturation monitoring method according to claim 1, further comprising at least the following steps after calculating the blood oxygen saturation of the infant by the formula:
E, determining a sample size of blood oxygen saturations of infants, and sample distribution and analysis data by statistical software according to clinical characteristics of the soles of infants and in combination with multi-center test requirements of clinical tests and linear regression in statistics; and
F, performing a normality test on the sample distribution and calculating a skewness.

7. An intelligent monitoring device using the infant blood oxygen saturation monitoring method according to claim 1, comprising a housing, and a thermometer and a blood oxygen heart rate monitor that are capable of being accommodated in the housing, wherein the thermometer is configured to monitor a body temperature under an armpit of an infant; the blood oxygen heart rate monitor is configured to be placed on a sole of the infant to monitor a blood oxygen heart rate; the housing is provided with a main control unit configured for signal interaction with the outside; and the thermometer and the blood oxygen heart rate monitor are in signal connection with the main control unit.

8. The intelligent monitoring device according to claim 7, wherein the test constants As, Bs, and Cs are calculated by the following steps:
C1, obtaining test data of a direct current component and an alternating current component corresponding to a blood oxygen value of 60 to 100 by a blood oxygen saturation calibrator; and
C2, in combination with clinical experience of a correspondence between blood oxygen values and blood flow signals at soles of an infant, calculating As, Bs, and Cs by combined polynomial fitting and nonlinear least square fitting.

9. The intelligent monitoring device according to claim 7, wherein in step B, I_{AC}^{λ1} and I_{AC}^{λ2} are calculated by:
I_{AC}^{λ1}=I_{PAC}^{λ1}=(I_{MAXAC}^{λ1}+I_{MINAC}^{λ1})/2; and
I_{AC}^{λ2}=I_{PAC}^{λ2}=(I_{MAXAC}^{λ2}+I_{MINAC}^{λ2})/2;
wherein I_{MAXAC}^{λ1} represents a maximum of an alternating current signal I_{DBAC}^{λ1} of a light signal with a wavelength of λ1 after being subjected to adaptive weighting and windowing and envelope detection;
I_{MINAC}^{λ1} represents a minimum of the alternating current signal I_{DBAC}^{λ1} of the light signal with the wavelength of λ1 after being subjected to adaptive weighting and windowing and envelope detection;
I_{MAXAC}^{λ2} represents a maximum of an alternating current signal I_{DBAC}^{λ2} of a light signal with a wavelength of λ2 after being subjected to adaptive weighting and windowing and envelope detection; and
I_{MINAC}^{λ2} represents a minimum of the alternating current signal I_{DBAC}^{λ2} of the light signal with the wavelength of λ2 after being subjected to adaptive weighting and windowing and envelope detection.

10. The intelligent monitoring device according to claim 9, wherein in step B, I_{DBAC}^{λ1} and I_{DBAC}^{λ2} are specifically calculated by the following steps:
B1, according to characteristics of the soles of an infant, sampling, as a basis, data of 30 pulse periods by the red light and the infrared light, respectively, with a sampling frequency of 100 Hz, to obtain about 3000 sample points I_{DBAC}^{λ1}[i], wherein i represents a serial number of a sample point, and i=1 to 3000; and
B2, substituting the data of the sample points into the following formula Y: RC*(I_{DBAC}^{λ1}[i]−I_{DBAC}^{λ1}[i−1])/Δt, to obtain I_{DBAC}^{λ1}[i]=I_{DBAC}^{λ1}[i−1]*[K/(K+1)], wherein K=RC/Δt.

11. The intelligent monitoring device according to claim 7, wherein in the formula X, values of the blood flow signals I_{MAX}^{λ1} and I_{MAX}^{λ2} are replaceable by direct current components I_{DC}^{λ1} and I_{DC}^{λ2} of the blood flow signals of λ1 and λ2, respectively, and calculated respectively by:
I_{DC}^{λ1}=I_{MAX}^{λ1}−I_{AC}^{λ1}; and
I_{DC}^{λ2}=I_{MAX}^{λ2}−I_{AC}^{λ2}.

12. The intelligent monitoring device according to claim 7, further comprising at least the following steps after calculating the blood oxygen saturation of the infant by the formula:
E, determining a sample size of blood oxygen saturations of infants, and sample distribution and analysis data by statistical software according to clinical characteristics of the soles of infants and in combination with multi-center test requirements of clinical tests and linear regression in statistics; and
F, performing a normality test on the sample distribution and calculating a skewness.
